(19) United States
(12) Patent Application Publication
(10) Pub. No.: US 2014/0243565 A1
(43) Pub. Date: Aug. 28, 2014

(54) PROCESS FOR PRODUCING BIOHYDROCARBONS

(71) Applicant: BASF SE, Ludwigshafen (DE)

(72) Inventor: Alexander Weck, Freinsheim (DE)

(73) Assignee: BASF SE, Ludwigshafen (DE)

(21) Appl. No.: 14/187,987

(22) Filed: Feb. 24, 2014

Related U.S. Application Data
(60) Provisional application No. 61/769,210, filed on Feb. 26, 2013.

(51) Int. Cl. C07C 4/06; C07C 1/22

(52) U.S. Cl. CPC C07C 4/06; C07C 1/22
USPC 585/251

(57) ABSTRACT

A process for producing biohydrocarbons, comprising the steps of

a) total hydrogenation of animal and/or vegetable oils, fats or mixtures thereof, forming propane from the glycerol component of the oils, fats or mixtures thereof and the corresponding alkanes from the fatty acid component of the oils, fats or mixtures thereof,

b) cracking the hydrocarbons obtained in step a) by thermal cracking, catalytic cracking or hydrocracking to form the biohydrocarbons.

The invention relates to processes for producing biohydrocarbons and to the use of totally hydrogenated animal and/or vegetable oils as a feedstock for a cracker.

The chemical industry is characterized by multistage value addition chains which are heavily branched. Starting materials are, for example, air, natural gases, naphtha, o-phosphate, potash, vacuum residues, rock salt, sulfur, benzene, cyclohexane and o-xylene. Products are, for example, propanol, ethanolamine, diethanolamine, C12-14-olefins, C13-15-alcohols, glycol ether, styrene/butadiene copolymers, polyisobutene, butyrolactone, tetrahydrofuran, methyl acrylate, dimethylacetamide, acrylic esters, butyraldehydes, butanols, ketenes, carbonic acid, melamine, polyvinyl chloride, propylene glycols, sodium nitride and sodium nitrate, sodium sulfite, sodium bisulfite, hydrosulfite, adipic acid/hexamethylenediamine salt, adipic acid and caprolactam. These products are used for further processing to end products.

The use of renewable raw materials in this integrated system is particularly advantageous when it is done at the start of the value addition chains, because existing structures can thus continue to be utilized, and the substantial networking of the last level which produces the end products makes it possible to attain all products through fewer reactants. End products are, for example, waxes, surfactants, plasticizers, polymers, solvent, adhesives, dispersions etc.

This multiplication effect makes the use of renewable substitutes for main inputs of naphtha, natural gas and vacuum residue particularly attractive, since the renewable raw materials are used further within the overall value addition chain.

While direct substitutes, such as vegetable oils for vacuum residue and biogas for natural gas, are suitable for production of synthesis gas, hydrogen and carbon monoxide, naphtha, for example for steamcrackers, cannot be replaced fully by oxygen compounds such as oils and fats of vegetable and animal origin, because the oxygenates formed to a considerable degree as a result of the oxygen input lead to severe corrosion phenomena on plant parts and cause process disruption extending as far as blockage of pipelines. Moreover, especially the unsaturated fatty acids which occur in natural fats and oils have a tendency to form resins and therefore significantly accelerate soling and fouling in the introductory stages of the overall process.

Different ways of reducing fouling are already known.

EP-A-2 290 034 describes the use of fatty acids in the feedstock of a steamcracker. These fatty acids are obtained by removing the liquid fractions of fats and oils from the solid fraction by steam distillation or vacuum distillation. Beforehand, a hydrolysis of the fats was conducted and glycerol was removed. In addition, an upstream partial hydrogenation can be performed to convert the double bonds to saturated bonds in the fatty acids. The hydrogenation can also be performed directly on the triglycerides.

WO 2011/012439 describes a process in which a complex mixture of natural fats and oils is first refined to remove non-triglycerides and non-fatty acids, then, for example, is hydrodeoxygenated, and subsequently, after a separation step, is optionally conducted into a steamcracker.

EP-A-2 290 045 describes a process for preparing biodiesel and bionaphtha and optionally biopropane from a complex mixture of natural fats and oils, wherein the natural fats and oils are first separated into liquid and solid triglycerides, the liquid triglycerides are converted to alkyl esters by transesterification, and the solid triglycerides are converted to linear or essentially linear paraffins by hydrodeoxygenation. It is also possible first to form the free fatty acids, which are then converted by hydrodeoxygenation or decarboxylation.

The fatty acids can be obtained by physical methods, such as steam distillation or vacuum distillation of the fats and oils, or else by hydrolysis of the triglycerides or acid hydrolysis of soaps.

Thus, a substantial pretreatment and division of the oils before and after the hydrogenation is needed, and so the process overall is costly and inconvenient and burdened with waste streams.

US 2007/0015947 A1 relates to a process for catalytic cracking of biorenewable feedstocks, wherein the feedstock is freed of impurities in an upstream step, for example by contacting with an acidic ion exchange resin. The feedstock is then subjected to the FCC cracking.

US 2009/0084026 A1 relates to a process in which an oil originating from natural sources is first hydrolyzed to form free fatty acids, then the fatty acids are separated into monounsaturated fatty acids and saturated fatty acids and polyunsaturated fatty acids, the monounsaturated and saturated fatty acids are esterified and the polyunsaturated fatty acids are subjected to a hydrotreatment.

Thus, in both processes, a costly and inconvenient pretreatment of the feedstocks is likewise necessary.

It is an object of the present invention to provide a process for producing biohydrocarbons, in which animal and/or vegetable oils, fats or mixtures thereof are used, with conversion of a maximum proportion of the carbon present in the oils, fats or mixtures thereof to the biohydrocarbons and with no need to separate the oils, fats or mixtures thereof prior to use in a (steam)cracker.

The object is achieved in accordance with the invention by a process for producing biohydrocarbons, comprising the steps of

a) total hydrogenation of animal and/or vegetable oils, fats or mixtures thereof, forming propane from the glycerol component of the oils, fats or mixtures thereof and the corresponding, preferably straight-chain, alkanes from the fatty acid component of the oils, fats or mixtures thereof,

b) cracking the hydrocarbons obtained in step a) by thermal cracking, catalytic cracking or hydrocracking to form the biohydrocarbons.

The object is additionally achieved by the use of totally hydrogenated animal and/or vegetable oils, fats or mixtures thereof, the glycerol component of the oils, fats or mixtures thereof forming propane and the fatty acid component of the oil, fats or mixtures thereof forming the corresponding, preferably straight-chain, alkanes, as a feedstock for a cracker.

It has been found in accordance with the invention that total hydrogenation of animal and/or vegetable oils, fats or mixtures thereof affords a product stream which can be conducted without further workup or separation into a cracker and processed further therein.

In step a), the glycerol component of the oils, fats or mixtures thereof forms propane, and the fatty acid component of the oils, fats or mixtures thereof the corresponding, preferably straight-chain, alkanes.

The term “straight-chain alkanes” is understood to mean alkanes which are at least 90% by weight, preferably at least 95% by weight and especially at least 99% by weight composed of linear alkanes.

The straight-chain alkanes can, for example, already be sold and used on the market for fuels as biodiesel II. The mixtures of propane and alkanes obtained can be introduced into downstream cracking operations without any losses in yield and operational reliability.

It is particularly advantageous to integrate the hydrogenation of the oils and fats of animal and vegetable origin into the raw material feed of the cracker:

A separation of the reaction products can be dispensed with; the crude output from the hydrogenation consisting of small amounts of residual hydrogen, propane and the (long-chain) alkanes can be sent directly, i.e. without purification and/or removal steps, to the feed vaporization of the (steam)cracker.

The direct supply of the crude output from the total hydrogenation, without purification and/or removal steps, to the cracker makes it possible to utilize the full natural carbon content of the reactant for the downstream processing in the cracker and hence for the subsequent value addition chains. In the case of utilization of biodiesel II, the propane would be utilized in the biodiesel plant for the purpose of generating energy and not in a physical manner. Preference is given to the full utilization of the crude output from the hydrogenation.

For the hydrogenation, it is possible to use product-containing crude hydrogen from the cracker. This need not be worked up since contamination of the hydrogenation with its own products is uncritical.

Use of the totally hydrogenated animal and/or vegetable oils, fats or mixtures thereof in a highly networked integrated site for production of chemical intermediates and end products allows the advantages which arise therefrom, such as reduction in the raw material and energy demands and minimization of the occurrence of waste and disposal streams, to be realized.

If biogenic raw materials are introduced into such an integrated structure, these are distributed over the entire value addition chain after a small number of production steps.

According to the invention, the term “biohydrocarbon” is understood to mean those hydrocarbons whose carbon content originates entirely or predominantly from renewable raw materials on an animal or vegetable basis. More particularly, the carbon atoms present in the biohydrocarbon originate entirely or predominantly from animal and/or vegetable oils, fats or mixtures thereof. These raw materials can also be referred to as biogenic raw materials or renewable raw materials.

“Animal and/or vegetable oils, fats or mixtures thereof” are understood to mean products formed very predominantly from triglycerides of fatty acids. Suitable sources are listed, for example, in US 2007/0015947 in paragraph [0007]. In addition, reference may be made to EP-A-2 290 045, especially paragraphs [0005] and [0011].

The animal and/or vegetable oils, fats or mixtures thereof may originate from any sources. They can, for example, be extracted or obtained from biomass before use in step a). Corresponding processes for removal/extraction of the oils, fats or mixtures thereof are known. For example, reference may be made to the documents cited in the prior art at the outset.

Preferably, no chemical or physical refining operation is conducted prior to the total hydrogenation, more particularly no degumming, optionally neutralizing, bleaching and deodorizing, as described in WO 2011/012439. The animal and/or vegetable oils, fats or mixtures thereof are accordingly conducted into the total hydrogenation without prior chemical or physical refining of this kind by the sequences mentioned.

The biomass used for this purpose may originate from any suitable sources, for example from wild plants, crop plants such as cereals, corn, beets or vegetables, from microalgae, or mixtures thereof.

The term “total hydrogenation” means that the animal and/or vegetable oils or fats or mixtures thereof are hydrogenated to such an extent that only vanishingly small amounts, if any, of bound oxygen are present in the products. Moreover, only minor amounts, if any, of carbon-carbon double bonds are present in the products. Accordingly, the glycerol component of the oils, fats or mixtures thereof forms propane, and the fatty acid component of the oils, fats or mixtures thereof forms the corresponding straight-chain alkanes shortened by one carbon atom.

It has been found in accordance with the invention that the hydrogenation product thus obtained can be converted further in a cracker without further purification or removal steps, without impairing the working of the cracker.

The stream originating from step a), or the hydrocarbons originating from step a), can thus be introduced into the cracker alone or together with naphtha and/or natural gas and cracked in step b).

According to the invention, the total hydrogenation can be effected by known processes. The total hydrogenation is preferably performed over a catalyst selected from catalysts comprising copper, noble metals or mixtures thereof as catalytically active metals.

The cracking of the hydrocarbons obtained in step a) is effected by thermal cracking, catalytic cracking or hydrocracking. Corresponding cracking processes are common knowledge and are described, for example, in K. Weissermel, H.-J. Arpe, Industrielle Organische Chemie, Bedeutende Vor- and Zwischenprodukte [Industrial Organic Chemistry, Important Precursors and Intermediates], 3rd edition, 1990, chapter 3 on pages 63 to 80.

The catalytic cracking is also described as FCC (Fluid Catalytic Cracking), since it is usually performed in a fluidized bed or in a reactor with rising catalyst (riser cracking). Cracking catalysts used are typically zeolites.

The ACR process (Advanced Cracking Reactor) can also be performed as the cracking process.

Hydrocracking, a form of catalytic cracking in the presence of hydrogen, preferentially forms saturated branched hydrocarbons. In the hydrocracking operation, the feedstocks are simultaneously desulfurized and denitrified, i.e. refined.

The thermal cracking is performed without catalyst and generally leads to a high proportion of olefins. In order to lower the partial pressure of the hydrocarbons, an extraneous gas, usually steam, is frequently added to the hydrocarbon cut to be thermally treated. This process, referred to as steamcracking, is preferred in accordance with the invention. Preferably, step b) is accordingly performed in a steam cracker with formation of bioethylene, biopropylene and biobutenes, and bio-C4 cuts comprising biobutadiene and biobutanes.

A composition of a typical C₄ cut in steamcracking and catalytic cracking is described in Weissermel/Arpe on page 73 in table 3-3.

The inventive cracking in step b), especially the steamcracking, can be operated by any suitable known processes.

The hydrogen used for total hydrogenation in step a) may originate from any suitable sources. For example and with preference, the hydrogen used in step a) may originate at least partly or fully from step b) and comprise cracking products formed in the cracker. This is harmless to the process according to the invention since the cracking products are converted again in the cracker.

The product mixture obtained in step a) can likewise be conducted into the cracker without further workup, which constitutes a considerable process simplification.

In the process according to the invention, C₂₋₄-olefins are more preferably formed as biohydrocarbons. These products can be used in accordance with downstream “green chemistry”, so as to result in a green value addition chain. In this context, “green” means the use of renewable raw materials.

The invention is illustrated in detail by the example which follows.

### EXAMPLE

a) A mixture of vegetable oils based on rapeseed oil is subjected to catalytic total hydrogenation in a hydrogenation reactor with input of hydrogen, giving an alkane mixture which is free of oxygenates to the trace level and comprises propane and alkanes having the same carbon number as in the fatty acid residue of the oil,

b) the alkane mixture obtained in step a) is converted directly, i.e. without further workup steps, in a steamcracker to a mixture of predominantly ethylene, propylene and a C₄ cut comprising butanes, butenes and butadiene. For this step, reference may be made to the process described in EP-A-2 290 034 and the parameters specified therein.

1. A process for producing biohydrocarbons, comprising the steps of
a) total hydrogenation of animal and/or vegetable oils, fats or mixtures thereof, forming propane from the glycerol component of the oils, fats or mixtures thereof and the corresponding alkanes from the fatty acid component of the oils, fats or mixtures thereof,
b) cracking the product mixture obtained in step a) by thermal cracking, catalytic cracking or hydrocracking to form the biohydrocarbons.

2. The process according to claim 1, wherein the entire product mixture obtained in step a) is conducted into the cracker without further workup.

3. The process according to claim 1, wherein step b) is performed in a steamcracker to form bioethylene, biopropylene and biobutenes, and bio-C₄ cuts comprising biobutadiene and biobutanes.

4. The process according to claim 1, wherein the hydrogen used for total hydrogenation in step a) originates at least partly from step b) and may comprise cracking products formed in the cracker.

5. The process according to claim 1, wherein the animal and/or vegetable oils, fats or mixtures thereof are extracted from biomass prior to use in step a).

6. The process according to claim 5, wherein the biomass originates from wild plants, crop plants from microalgae, or mixtures of wild plants, crop plants, and/or microalgae.

7. The process according to claim 1, wherein the products originating from step a) are cracked together with gas oil, liquefied gas, refinery gases, naphtha and/or natural gas in step b).

8. The process according to claim 1, wherein the total hydrogenation is performed over a catalyst selected from catalysts comprising copper, noble metals or mixtures of copper and noble metals as catalytically active metals.

9. The process according to claim 6, wherein the crop plants are cereals, corn, beets or vegetables.
